(19) United States
(12) Patent Application Publication
(10) Pub. No.: US 2024/0412242 A1
(43) Pub. Date: Dec. 12, 2024

(54) DATABASE FILE MANAGEMENT SYSTEM FOR AN INTERACTIVE ARTIFICIAL INTELLIGENCE PERSONALIZED REWARDS SYSTEM

(71) Applicant: Richard Bruno Muller, Marietta, GA (US)

(72) Inventor: Richard Bruno Muller, Marietta, GA (US)

(21) Appl. No.: 18/786,431

(22) Filed: Jul. 26, 2024

Related U.S. Application Data
(63) Continuation-in-part of application No. 18/208,348, filed on Jun. 12, 2023, now abandoned.

(51) Int. Cl. G06Q 30/0207; G06Q 30/0226; G16H 50/30

(52) U.S. Cl. CPC G06Q 30/0215; G06Q 30/0233; G16H 50/30

(57) ABSTRACT

The present invention discloses a database file management system for an interactive artificial intelligence personalized rewards system including a plurality of databases, a network computer configured to gather personalized user data including user personal information, user transactional purchases, and user self-reported information for storage on the plurality of databases, a database schema configured to categorize the personalized user data, a mobile device with a camera configured to capture images of at least one document with health related information about the user, an inter-process communication processor configured to analyze the machine readable text to create a health risk assessment of the user and to analyze the health risk assessment and the personalized user data to determine potential third-party redemption benefits for the user, wherein the inter-process communication processor is further configured to assign customized third-party redemption benefits to the user based on the categorized personalized user data.

### CROSS-REFERENCE TO RELATED APPLICATIONS

This patent application is a continuation-in-part and claims priority to the United States patent application entitled: “DATABASE FILE MANAGEMENT SYSTEM”, U.S. Ser. No. 18/208,348 filed on Jun. 12, 2023, filed by Richard Bruno Muller, which is a continuation and claims priority to based on patented U.S. patent Ser. No. 17/588,323 filed on Jan. 30, 2022, entitled “DNA DATA PERSONALIZED MEMBER BENEFITS PROGRAM AND METHOD”, by Richard Bruno Muller, which is a continuation and claims priority to based on patented U.S. patent Ser. No. 16/271,801 filed on Feb. 9, 2019, entitled “REWARDS PROGRAM METHOD AND DEVICES”, by Richard Bruno Muller, the U.S. patent application and patent being incorporated herein by reference.

### BACKGROUND

Database management is in most cases beyond a normal computer user's experience level. While data is the primary means of receiving and sending information proper care and maintenance are needed. A system for database file management can make computer use more efficient and less problematic for the user.

### BRIEF DESCRIPTION OF THE DRAWINGS

FIG. 1 shows for illustrative purposes only an example of a database file management system of one embodiment.

FIG. 2 shows for illustrative purposes only an example of a database file management system hierarchy of one embodiment.

FIG. 3 shows for illustrative purposes only an example of a database management system of one embodiment.

FIG. 4 shows a block diagram of an overview flow chart of the rewards program method and devices of one embodiment.

FIG. 5A shows a block diagram of an overview of rewards program credit card cardholder members of one embodiment.

FIG. 5B shows a block diagram of an overview of contracting with companies in a rewards program of one embodiment.

FIG. 6 shows a block diagram of an overview of the rewards program digital network of one embodiment.

FIG. 7 shows a block diagram of an overview of the rewards program digital device application of one embodiment.

FIG. 8 shows for illustrative purposes only an example of a rewards program server of one embodiment.

FIG. 9 shows a block diagram of an overview of rewards program member memberships of one embodiment.

FIG. 10 shows for illustrative purposes only an example of self-reported data of activities of daily living of one embodiment.

FIG. 11 shows for illustrative purposes only an example of health risk assessment of one embodiment.

FIG. 12 shows for illustrative purposes only an example of tracking your social media of one embodiment.

FIG. 13 shows for illustrative purposes only an example of concierge travel plans of one embodiment.

FIG. 14 shows for illustrative purposes only an example of tracking your visits of one embodiment.

FIG. 15 shows a block diagram of an overview of AutoBuy earnest rewards program benefits of one embodiment.

FIG. 16 shows a block diagram of an overview of vehicle purchase and related benefits of one embodiment.

FIG. 17A shows a block diagram of an overview of community banking system participant benefits of one embodiment.

FIG. 17B shows a block diagram of an overview of the random drawing for members of one embodiment.

FIG. 18 shows a block diagram of an overview of an Opt.In Ecosystem program of one embodiment.

FIG. 19 shows a block diagram of an overview of the AutoBuy Earnfast rewards program worldwide of one embodiment.

FIG. 20 shows a block diagram of an overview of the AutoBuy Rewards program revenue streams of one embodiment.

FIG. 21 shows a block diagram of an overview of the AutoBuy Rewards program micro-payments continued of one embodiment.

FIG. 22 shows a block diagram of an overview of escrow account earned interest commissions of one embodiment.

FIG. 23 shows a block diagram of an overview of a plurality of databases of one embodiment.

### DETAILED DESCRIPTION OF THE INVENTION

In a following description, reference is made to the accompanying drawings, which form a part hereof, and which are shown by way of illustration a specific example in which the invention may be practiced. It is to be understood that other embodiments may be utilized and structural changes may be made without departing from the scope of the embodiments.

It should be noted that the descriptions that follow, for example, in terms of a rewards program method and devices are described for illustrative purposes and the underlying system can apply to any number and multiple types of credit card transactions and product and service benefits. In one embodiment of the present invention, the rewards program method and devices can be configured using member-accumulated points to determine benefits available to a member. The rewards program method and devices can be configured to include OEM manufacturer participants and can be configured to include credit card company participants using the embodiments.

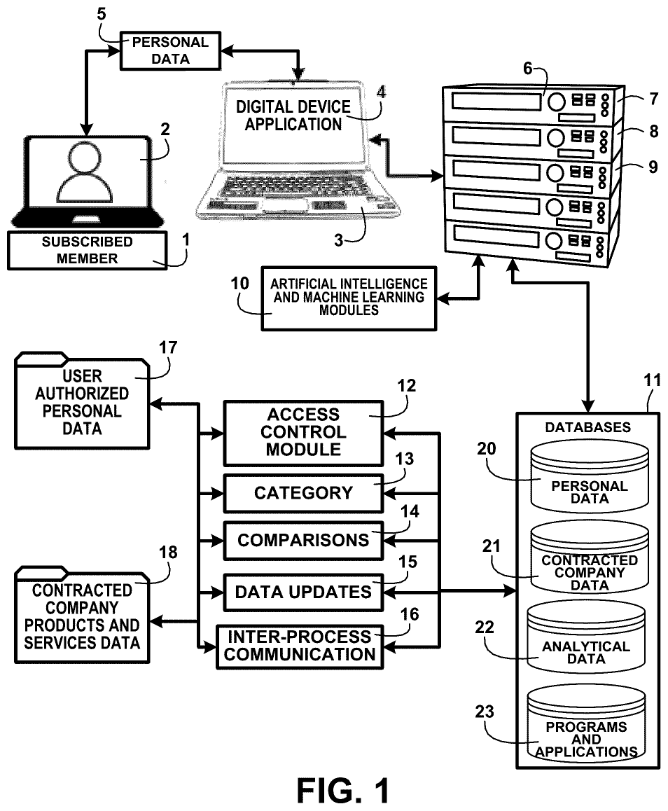

FIG. 1 shows for illustrative purposes only an example of a database file management system of one embodiment. FIG. 1 shows a subscribed member 1 with a subscribed member mobile device 2 having a digital device application 4 to enter personal data 5 to a network computer 3. Network 6 includes a plurality of servers 7, a plurality of digital processors 8, and a plurality of communication devices 9. A plurality of databases 11 coupled to the plurality of servers 7 stores the personal data 20, contracted company data 21, analytical data 22, programs and applications 23, human interactive artificial intelligence and machine learning modules 10, and access control module 12 confirms the subscribed member identification and the contracted company identification before allowing the login to enter and retrieve data from the network 6.

The subscribed member 1, which is a human, interacts with the artificial intelligence and machine learning modules 10 to receive personalize rewards to enhance the subscribed member's 1 engagement and satisfaction. In one embodiment, the artificial intelligence and machine learning modules 10 analyze the subscribed member's 1 input data, such as purchase history, browsing patterns, and social media interactions. The subscribed member 1 can identify individual preferences, likes, and dislikes. This allows for the creation of a tailored rewards program where each subscribed member 1 can receive offers and incentives that align with their specific interests. For instance, the artificial intelligence and machine learning modules 10 determines that the subscribed member 1 frequently purchases athletic gear, the artificial intelligence and machine learning modules 10 may prioritize offering discounts and purchasing rewards on sports-related items or exclusive access to fitness events. This personalized approach not only increases the likelihood of the subscribed member 1 engaging with the system, but also fosters a deeper sense of loyalty and connection to a brand. Over time, as the artificial intelligence and machine learning modules 10 continue to learn and adapt from subscribed member 1 behavior, the rewards system becomes increasingly accurate and effective, providing a continually evolving and highly customized user experience.\

The plurality of databases 11 provides access to the data stored for inter-process communication 16 to processes including a process to identify one or more category 13 of data sets into indexed data. The inter-process communication 16 further facilitates comparisons 14 of personal data 5 with contracted company data 21 to target advertisements stored in the contracted company data 21 that are related to personalized indexed personal data 5 to display to the subscribed member 1. The plurality of databases 11 additionally stores data updates 15 received in the network 6. The user-authorized personal data 17 is analyzed to determine using the contracted company products and services data 18 to correlate products and services data with subscribed member 1 personal data 5 demographics, health, daily activities for example. The results of the correlation are stored on the plurality of databases 11.

The interprocess communication 16 operates between programs, applications, and database data for the personalization of subscribed member personal data with contracted company products and services. Inter-process communication (IPC) is the mechanism an operating system provides to allow the processes to manage shared data. An IPC mechanism is either synchronous or asynchronous. Synchronization primitives may be used to have synchronous behavior with an asynchronous IPC mechanism of one embodiment.

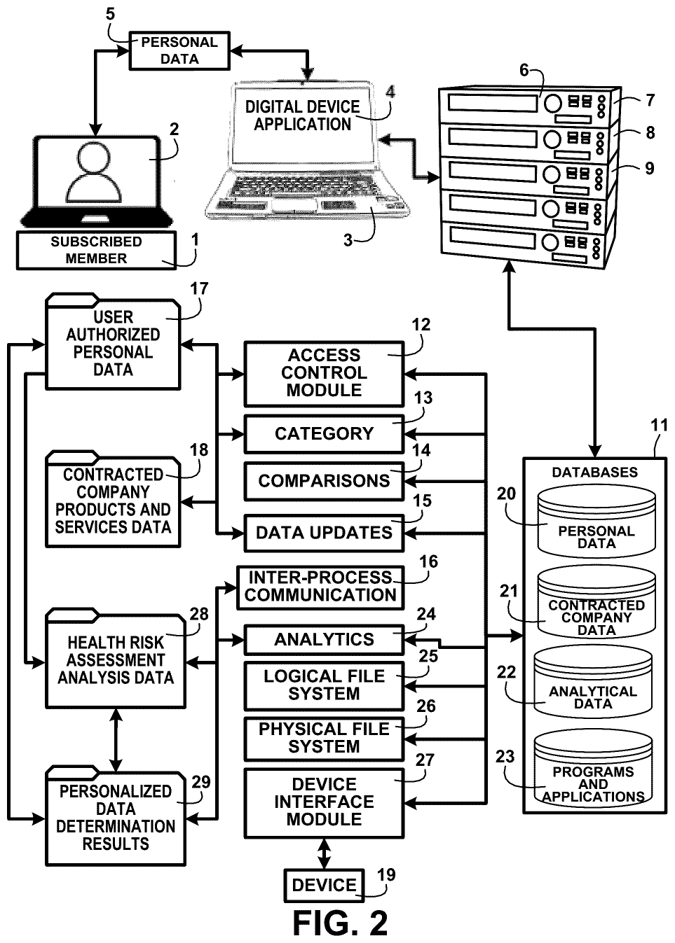

FIG. 2 shows for illustrative purposes only an example of a database file management system hierarchy of one embodiment. FIG. 2 shows the subscribed member 1 using a subscribed member mobile device 2 to enter personal data 5 to the network computer 3 with a digital device application 4. Network 6 includes a plurality of servers 7, a plurality of digital processors 8, and a plurality of communication devices 9. The plurality of databases 11 stores the entered personal data 20. Additionally the plurality of databases 11 stores contracted company data 21, and the results of analytical data 22. The plurality of databases 11 also stores programs and applications 23 for use in the overall database management system. Access to the data stored on the plurality of databases 11 is processed with an access control module 12 to confirm logins and queries are from authorized entries for that specific data. The processes to analyze category 13 data indexes, make comparisons 14 between sets of indexed data, and record on the plurality of databases 11 data updates 15 are in part processed with inter-process communication 16. For example, comparisons of user-authorized personal data 17 and contracted company products and services data 18 run analysis of one set of data to the other to determine commonalities.

This inter-process communication between programs, applications, and database data for the personalization of subscribed member personal data with contracted company products and services results in the identification of contracted company products and services closely related to the subscribed member's personalized personal data. The various analytics 24 results are recorded in the plurality of databases 11. The logical file system 25 and physical file system 26 processes the stored and incoming data for organizing the data for faster query responses. Analytics 24 results include for example health risk assessment analysis data 28 determined from the personal data 5 and personalized data determination results 29 are stored in the plurality of databases 11 and indexed for queries. A device interface module 27 stores device drivers to operate device 19 coupled to the network of one embodiment.

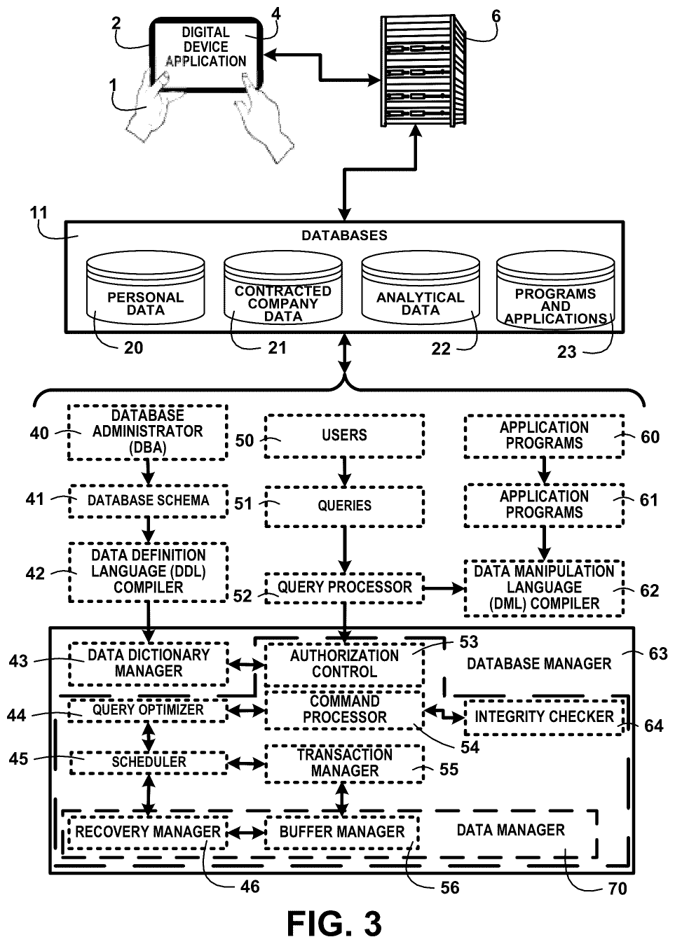

FIG. 3 shows for illustrative purposes only an example of a database management system of one embodiment. FIG. 3 shows the subscribed member 1 using the subscribed member mobile device 2 having the digital device application 4 communicating with the network 6. The plurality of databases 11, database for the management system to perform the operations to maintain, update and preserve the data for processes and queries 51 by users 50.

The database management system includes a database manager 63 and is a gatekeeper of the personal data 20, contracted company data 21, analytical data 22, and programs and applications 23. Database manager 63 implements security programs to ensure the safety of database-stored data. A database administrator (DBA) 40 manages the database schema 41. The database administrator (DBA) is responsible for maintaining, securing, and operating databases and also ensures that data is correctly stored and retrieved. In addition, DBAs often work with developers to design and implement new features and troubleshoot any issues.

The database schema defines how data is organized within a relational database, this is inclusive of logical constraints such as table names, fields, data types, and the relationships between these entities. Schemas commonly use visual representations to communicate the architecture of the database, becoming the foundation for an organization's data management discipline. This process of database schema design is also known as data modeling.

The database management system includes a data definition language (DDL) compiler 42. The DDL compilers used to create and modify the structure of objects in a database using predefined commands and a specific syntax. A data dictionary manager 43 records names and nomenclature of the data for ease in queries for the data associated with the name or nomenclature. The database management system utilizes a query optimizer 44. The query optimizer is cost-based. Where cost, is the amount of time to respond to a query. It analyzes several candidate execution plans for a given query, estimates the cost of each of these plans, and selects the plan with the lowest cost of the choices considered. This has an impact on the performance of the databases. A scheduler 45 is included to store in the databases its persistent information and store tasks and then run them. A recovery manager 46 of the database management system captures the present state of the database) and records that in a log on the database, to aid in a recovery.

The database management system provides users 50 with the capability to make queries 51 through a query processor 52. The user query first is processed through authorization control 53 to confirm the identity of the user as authorized with permission to access the data. A command processor 54 is a program that receives control when a user at a terminal enters a command name. A transaction manager 55 is a logical unit of processing in a database management system that entails one or more database access operations. It is a transaction program unit whose execution may or may not change the contents of a database.

A buffer manager 56 of a data manager 70 is used to manage the allocation of the space available in the main memory for the storage of blocks. A listing of application programs 60 is displayed for the application programs 61 stored on the plurality of databases 11 from the programs and applications 23 databases. A data manipulation language (DML) compiler 62 processes DML statements used to work with the data in tables. An integrity checker 64 is a database configuration utility that you can use to assess the health of the base layer data dictionary of one embodiment.

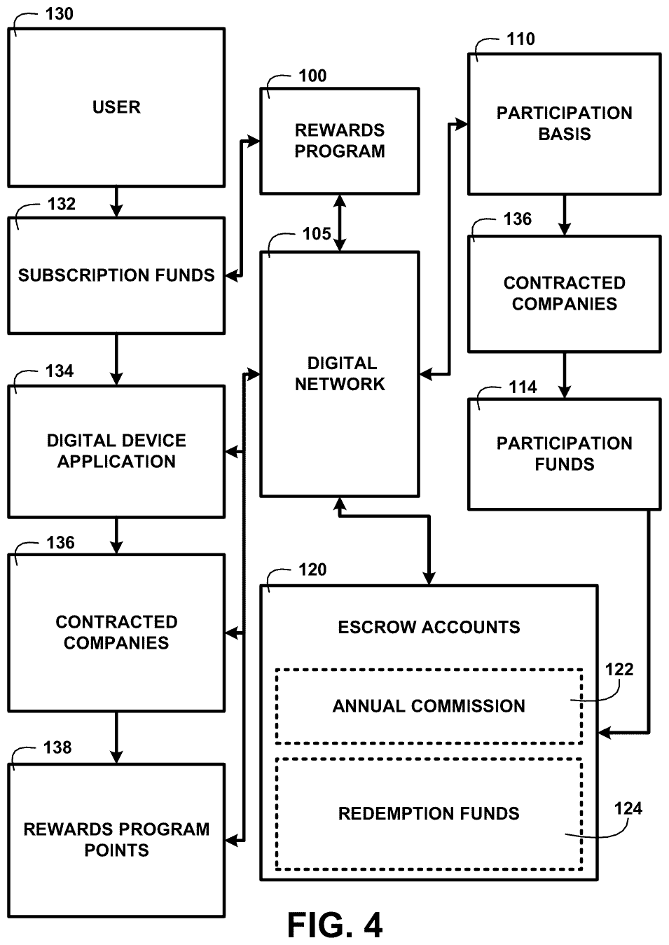

FIG. 4 shows a block diagram of an overview flow chart of the rewards program method and devices of one embodiment. FIG. 4 shows establishing a rewards program 100 using a rewards program digital network 105 for receiving member data and providing interactive data services and functions. Establishing a rewards program 100 includes contracting with companies on a participation basis 110 in a rewards program. Contracted participating companies will execute a first 5 years Agreement/Contract; a second 5 years Agreement/Contract; a third 5 years Agreement/Contract; and one (1) 35 years Contract/Agreement of one embodiment.

Funding the rewards program includes receiving contracted companies 136 participation funds. Depositing contracted companies' participation funds 114 allows establishing CMERF and VRFPI program interest-earning escrow accounts 120. Earning interest with the Car Manufacturers Escrow Redemption Fund (CMERF) and Vendor Reward Fund Participation Income (VRFPI) escrow accounts provides income to the rewards program company for operating by paying rewards program earned interest annual commission 122 to the rewards program company. Paying rewards program subscribed member redemption funds 124 for purchases from contracted companies using the balance of the CMERF and VRFPI program escrow accounts interest earnings can increase member purchases from contracted participating companies.

The rewards program includes enrolling credit card cardholder members user 130 in a subscription usage-based pricing rewards program. Enrolling credit card cardholder members in a subscription usage-based pricing rewards program user 130 further is increasing income through receiving credit card cardholder member subscription funds 132. The rewards program can personalize member rewards benefits by personalizing member data authorized by members via a rewards digital device application 134.

Learning member likes and dislikes and daily living activities can include for example a member's health condition when providing personalized data used to personalize a member's benefits. Benefits come from earning rewards program points by subscribed members with purchases from contracted companies 136. Redeeming rewards program points by subscribed members purchasing from the contracted companies 138 can reduce subscribed members out of pocket purchasing expenses of one embodiment.

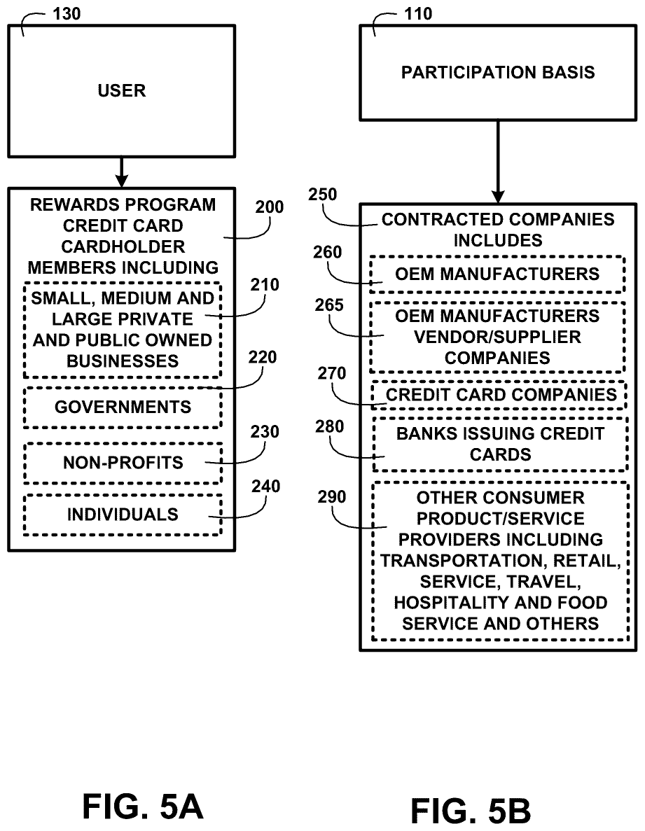

FIG. 5A shows a block diagram of an overview of rewards program credit card cardholder members of one embodiment. FIG. 5A shows enrolling credit card cardholder members in a subscription usage-based pricing rewards program user 130. Enrollment of rewards program credit card cardholder members including 200 small, medium, and large private and public-owned businesses 210, governments 220, non-profits 230 and individuals 240 can lead to a rapid growth of member enrollment of one embodiment.

FIG. 5B shows a block diagram of an overview of contracting with companies in a rewards program of one embodiment. FIG. 5B shows contracting with companies in a rewards program on a participation basis 110 increases the range of contracted company participation funds and member benefits offerings. The rewards program participation contracted companies include 250 OEM manufacturers 260, OEM manufacturers vendor/supplier companies 265, credit card companies 270, banks issuing credit cards 280, and other consumer product/service providers including transportation, retail, service, travel, hospitality, and food service and others 290 of one embodiment.

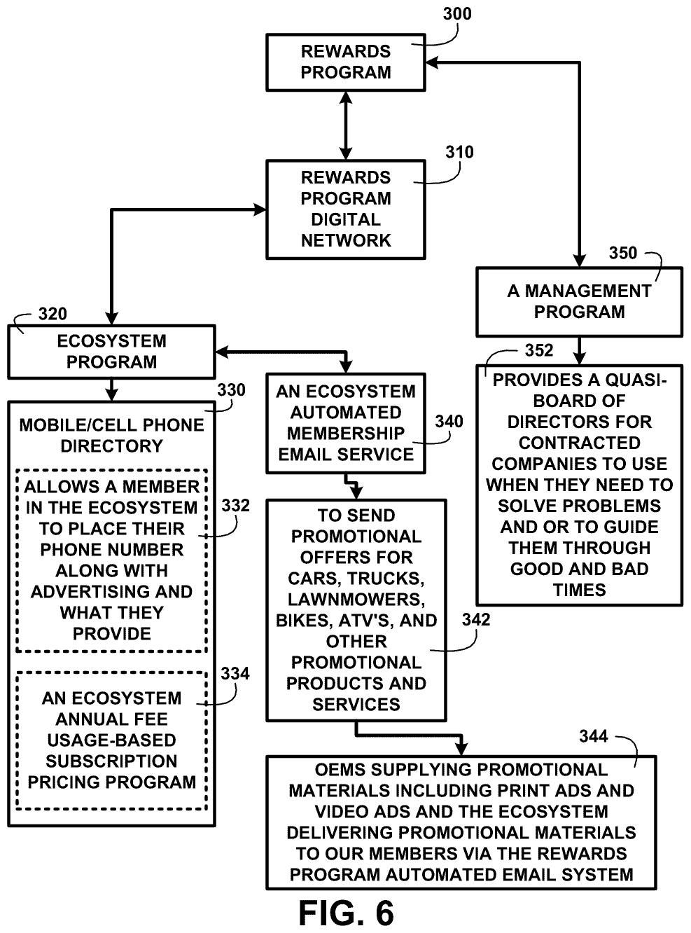

FIG. 6 shows a block diagram of an overview of the rewards program digital network of one embodiment. FIG. 6 shows a rewards program 300 including a rewards program digital network 310. The rewards program digital network 310 includes an Ecosystem program 320 including a mobile/cell phone directory 330. The mobile/cell phone directory 330 allows a member in the Ecosystem to place their phone number along with advertising and what they provide 332. The Ecosystem program 320 includes an Ecosystem annual fee usage-based subscription pricing program 334. The rewards program digital network 310 includes an Ecosystem automated membership email service 340 to send promotional offers for cars, trucks, lawnmowers, bikes, ATVs, and other promotional products and services 342 to subscribed members. The rewards program digital network 310 includes OEMs supplying promotional materials including print ads and video ads and Ecosystem 344 delivers promotional materials to our members via the automated membership email service. The rewards program digital network 310 includes a management program 350 element which provides a quasi-board of directors for contracted companies to use when they need to solve problems and or to guide them through good and bad times 352 of one embodiment.

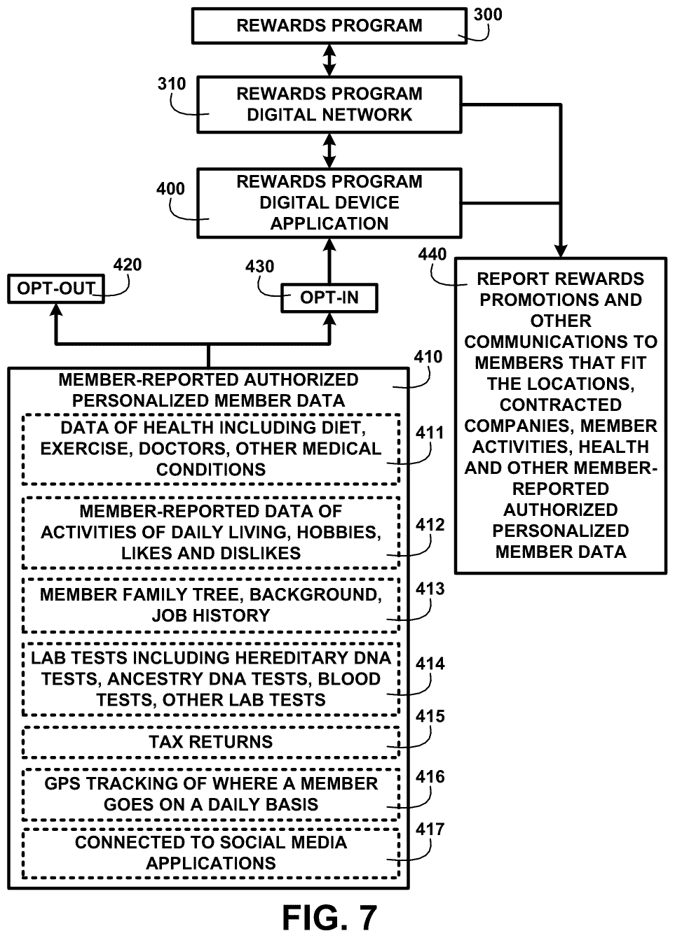

FIG. 7 shows a block diagram of an overview of the rewards program digital device application of one embodiment. FIG. 7 shows the rewards program 300 rewards program digital network 310 including a rewards program digital device application 400. The rewards program digital device application 400 can be used to report rewards promotions and other communications to members that fit the locations, contracted companies, member activities, health, and other member-reported authorized personalized member data 440.

A subscribed member may choose an opt-out 420 selection to not authorize member-reported personalized member data using the rewards program digital device application 400. The subscribed member may choose an opt-in 430 selection to authorize the gathering of member-reported authorized personalized member data 410 using the rewards program digital device application 400. Authorized personalized member data can include data on health including diet, exercise, doctors, and other medical conditions 411, member-reported data of activities of daily living, hobbies, likes and dislikes 412, member family tree, background, job history 413, lab tests including hereditary DNA tests, ancestry DNA tests, blood tests, other lab tests 414, tax returns 415, and GPS tracking of where a member goes daily 416. The member authorization can include the rewards program digital device application 400 to be connected to social media applications 417 of the subscribed member of one embodiment.

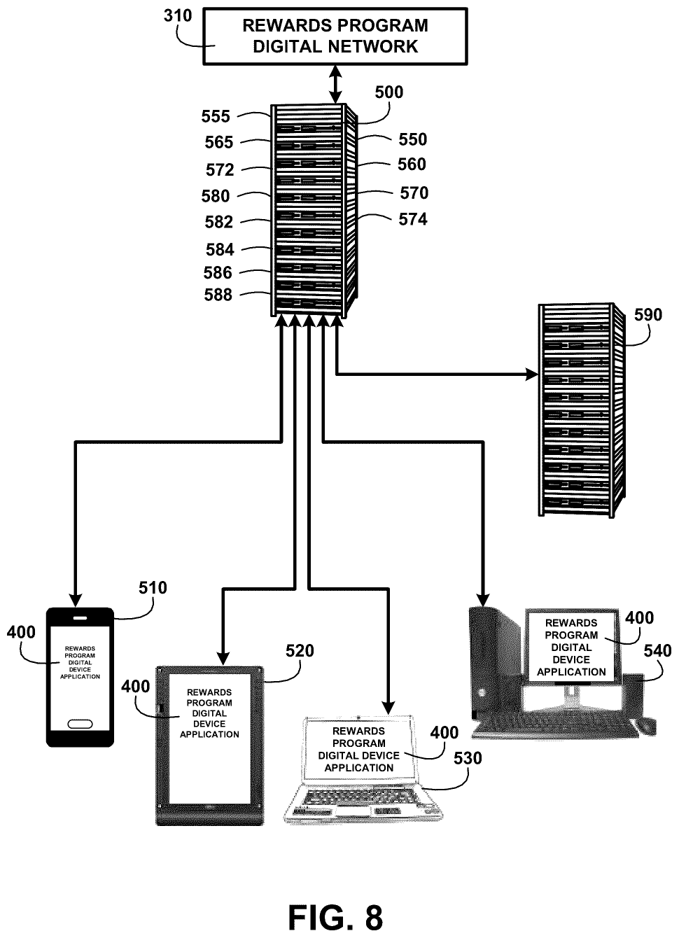

FIG. 8 shows for illustrative purposes only an example of a rewards program server of one embodiment. FIG. 8 shows the rewards program digital network 310 including at least one rewards program server 500; at least one digital database 550; at least one digital processor 560; at least one OCR device 570; at least one WI-FI connectable device 574; at least one printer 555; at least one video device 565; at least one language translation apparatus 572; at least one data mining device 580; at least one internet connectable device 582; and voice recognition and transcription device 584; at least one email server 588 and an automated membership email service database 586 of one embodiment.

At least one digital database 550 can be used for example to record and retrieve authorized personalized member data. At least one digital processor 560 can be used for example for processing authorized personalized member data to analyze the personalized member data for example to determine a subscribed member's health risk assessment. At least one OCR device 570 can be used for example for converting a subscribed member's ancestry DNA tests to a readable text for use in determining the subscribed member's health risk assessment. At least one WI-FI connectable device 574 can be used for example for connecting a user's digital device to at least one rewards program server 500 for gathering authorized personalized member data with the rewards program digital device application 400 of one embodiment.

At least one printer 555 can be used for example for printing hard copy reports for a contracted company of VRFPI program escrow accounts balances and transactions. At least one video device 565 can be used for example for recording promotional materials for distribution to subscribed members using the mobile/cell phone directory 330 and the rewards program automated email system 344. At least one language translation apparatus 572 can be used for example for translating contracted companies' promotional materials into a subscribed members' selected default language of one embodiment.

At least one data mining device 580 can be used for example for gathering up-to-date information and data on hundreds of brands and models for subscribed member buyers to choose from for redemption of reward points purchases. At least one internet-connectable device 582 can be used for example for connecting a user's digital device with the rewards program digital device application 400 installed for browsing reward points redemption opportunities of one embodiment.

The rewards program digital network 310 including at least one rewards program server 500 can include an interactive connection to at least one contracted company server 590. The interactive connection to at least one contracted company server 590 can be used for reporting subscribed member purchases; redemption points and participation funds transactions; CMERF and VRFPI program escrow account balances and transactions; transfers of promotional materials and other communications and data transfers of one embodiment.

The rewards program digital network 310 at least one rewards program server 500 can provide an interactive connection for example for subscribed members transactions using subscribed member's digital devices including a smartphone 510 with the rewards program digital device application 400 installed; a tablet 520 with the rewards program digital device application 400 installed; a laptop computer 530 with the rewards program digital device application 400 installed; and a computer 540 with the rewards program digital device application 400 installed of one embodiment.

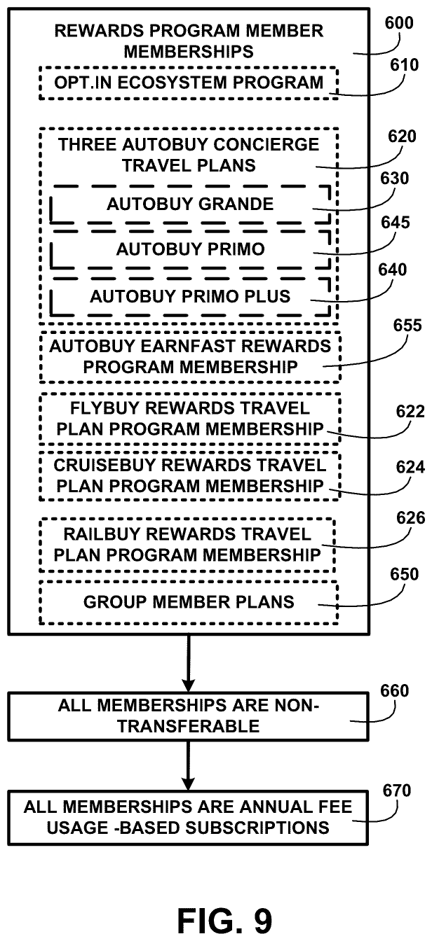

FIG. 9 shows a block diagram of an overview of rewards program member memberships of one embodiment. FIG. 9 shows rewards program member memberships 600 that a subscribed member can select including an Opt.In Ecosystem program 610; three AUTOBUY concierge travel plans 620 including AUTOBUY Grande 630, AUTOBUY PRIMO 645, and AUTOBUY PRIMO PLUS 640; AutoBuy Earnfast Rewards Program membership 655; and flybuy rewards travel plan program membership 622, cruisebuy rewards travel plan program membership 624, railbuy rewards travel plan program membership 626; and group member plans 650 and other member plans. All memberships are non-transferable 660. All memberships are annual fee usage-based subscriptions 670 of one embodiment.

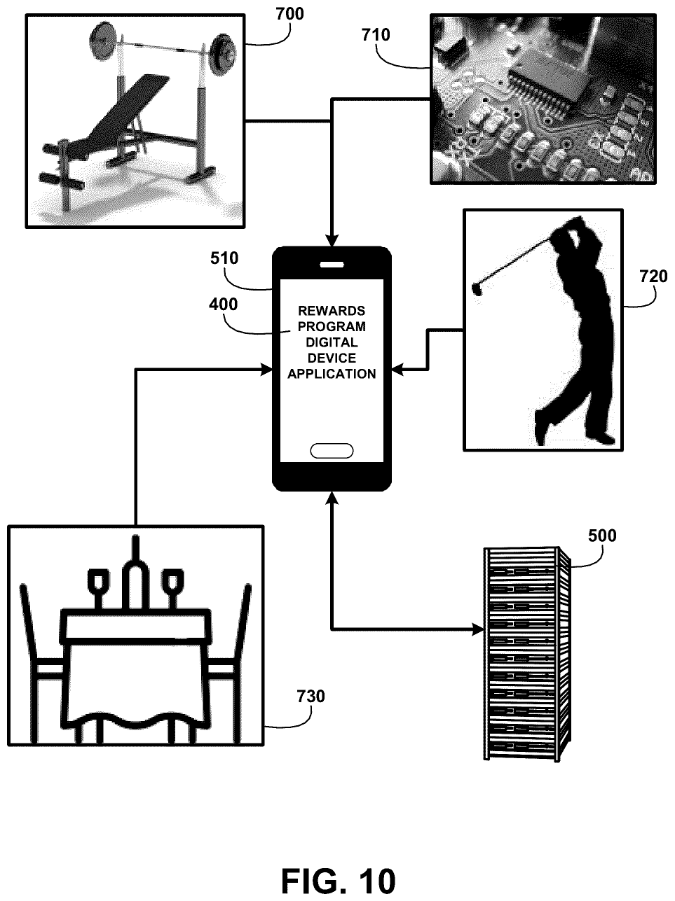

FIG. 10 shows for illustrative purposes only an example of self-reported data OF activities of daily living of one embodiment. FIG. 10 shows the subscribed member smartphone 510 with the rewards program digital device application 400 installed. The rewards program digital device application 400 is uploading to at least one rewards program server 500 the subscribed members' activities including for example going to a fitness club 700; participating in an electronics hobby 710; playing golf 720 and enjoying fine dining 730 at an upscale restaurant. The rewards program digital device application 400 is collecting authorized opt-in self-reported data including the subscribed members' activities of daily living, hobbies, likes, and dislikes. The rewards program digital device application 400 can also be used for collecting the member's tax returns and performing GPS tracking of where the member is going daily of one embodiment.

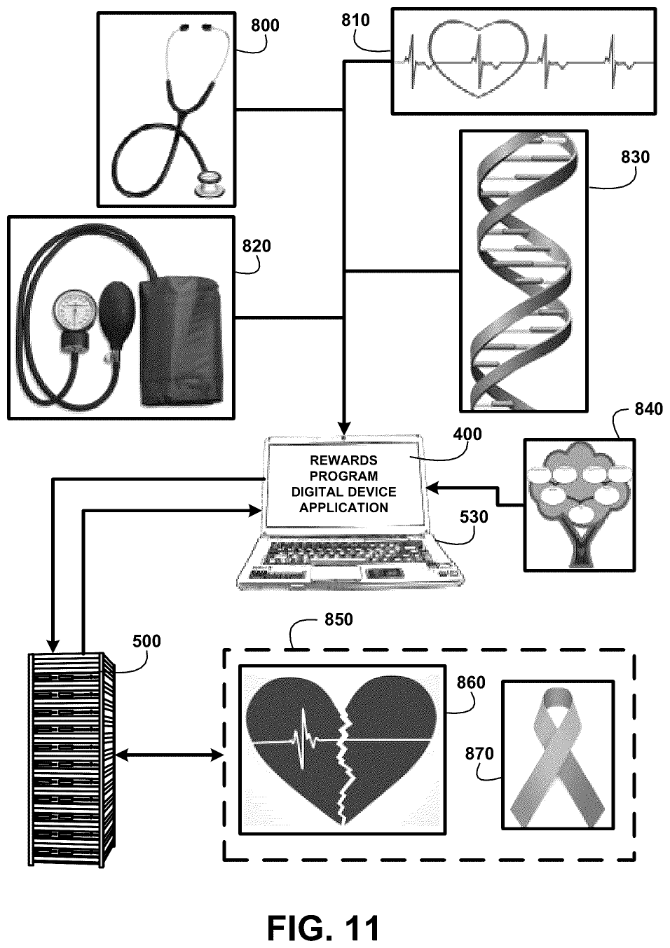

FIG. 11 shows for illustrative purposes only an example of health risk assessment of one embodiment. FIG. 11 shows the subscribed members' laptop computer 530 with the rewards program digital device application 400 installed communicating with at least one rewards program server 500 via at least one internet connectable device 582. The subscribed member can self-reporting their health condition 800; cardio lab tests 810; blood pressure 820; DNA results 830; and family tree and history 840; background, for example, any military duty, or job history which for example may indicate potential exposure to hazardous materials, other lab tests and hereditary DNA tests, ancestry DNA tests, and blood tests.

The at least one rewards program server 500 using at least one digital processor 560 can perform a subscribed members' health risk assessment 850 using the self-reported health condition 800 and other health-related data to perform a heart condition analysis 860 based on the DNA results 830; family tree and history 840; blood pressure 820 and cardio lab tests 810, background, for example, any military duty, job history which for example may indicate potential exposure to hazardous materials, other lab tests and hereditary DNA tests, ancestry DNA tests, and blood tests. At least one rewards program server 500 using at least one printer 555 can print the heart condition analysis 860 for example in a .pdf format and email the results to the subscribed member.

The at least one rewards program server 500 using at least one digital processor 560 can perform a subscribed members' health risk assessment 850 using the self-reported health condition 800 and other health-related data to perform a breast cancer analysis 870 based on the DNA results 830; and family tree and history 840; background, for example, any military duty, job history which for example may indicate potential exposure to hazardous materials, other lab tests and hereditary DNA tests, ancestry DNA tests, and blood tests. At least one rewards program server 500 using at least one printer 555 can print the breast cancer analysis 870 for example in a hard copy format and mail the results to the subscribed member for example for the member to share with their physician. The health risk assessment personalized for the subscribed member may provide them with early health risks alerts they were unaware of and could lead to early detection and treatment of a potentially serious health condition of one embodiment.

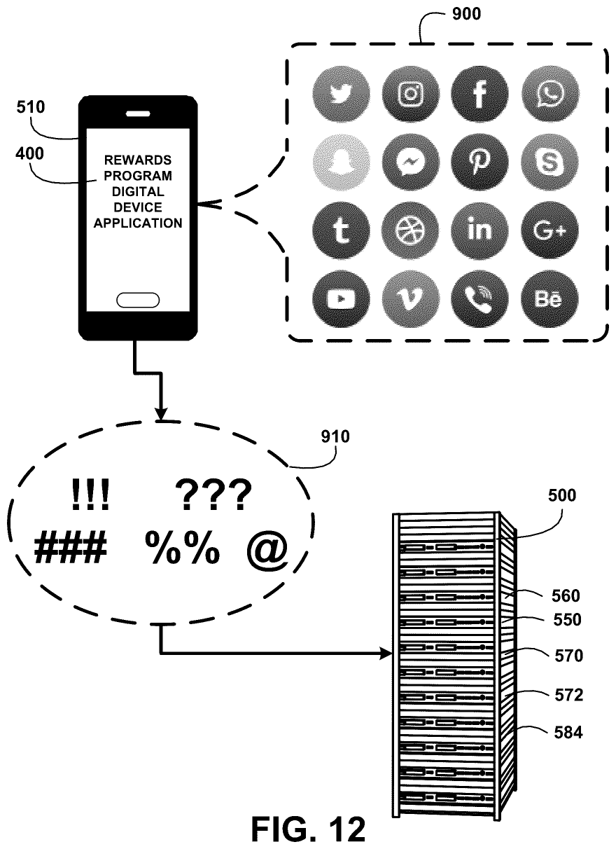

FIG. 12 shows for illustrative purposes only an example of tracking your social media of one embodiment. FIG. 12 shows the subscribed members' smartphone 510 with the rewards program digital device application 400 installed providing a connection with at least one rewards program server 500 for collecting authorized social media dialogues 900 and social media comments 910 of the member and their families, friends, and associates. Tracking a member's social media provide an analysis opportunity to determine what areas a member and their friends talk about and debate about. The social media analysis can pinpoint additional likes and dislikes of a subscribed member to direct for example rewards points redemption opportunities that fit into their personalized likes and dislikes and potential bucket list interests.

The social media analysis can be performed by at least one rewards program server 500; at least one digital database 550 using recorded member personalized data; at least one digital processor 560 analytics of the at least one OCR device 570 parsed content of the social media subjects talked about and debated about and recorded in the database using the voice recognition and transcription device 584. At least one language translation apparatus 572 can be used to confirm the specific meaning of a term or phrase in a language to arrive at any difference associated with a local nuance ascribed to that particular term or phrase of one embodiment.

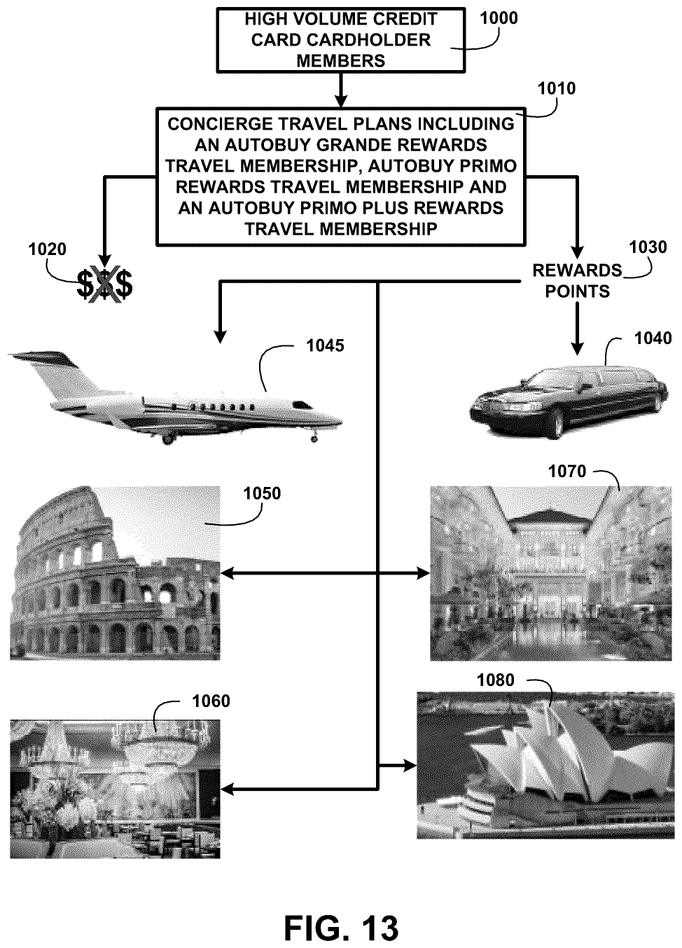

FIG. 13 shows for illustrative purposes only an example of concierge travel plans of one embodiment. FIG. 13 shows high volume credit card cardholder members 1000 may accumulate adequate AutoBuy rewards points for redemption using one of the AutoBuy rewards concierge travel plans including an AutoBuy Grande Rewards Travel Membership; AutoBuy Primo Rewards Travel Membership; and an AutoBuy Primo Plus Rewards Travel Membership 1010. The rewards travel memberships are subscription plans wherein a subscribed member can enjoy reduced to no cash expense 1020 and use rewards points 1030 to purchase contracted company travel opportunities. For example, a rewards travel member can use points accumulated to pay the expenses of Shared Private Jet transportation travel 1045 including international travel 1050. Rewards travel benefits can also include ground transportation 1040 including limousine service; hotel accommodations 1070; fine dining 1060; and entertainment 1080 at world-class events and facilities of one embodiment.

These three special memberships are only available when redeeming AutoBuy Reward Points. If the Shared Private Jet travel exceeds a member's AutoBuy Reward Points they can pay the balance with any contracted credit card. These three Concierge Memberships are designed to be used by families who travel regularly for either business or pleasure. These are also designed for small, medium, and large businesses, corporations, and governments that don't have a personal or corporate agency or travel department. These memberships are designed to provide access to a Rewards program professional concierge. These professionals will assist you in securing personal or business services, planning a family trip, and securing plane flights with one of the Rewards programs Shared Private Jet partner firms.

AutoBuy GRANDE Rewards Travel members can access a professional concierge 16 hours a day 365 days a year. The annual subscription fee is payable by a single or double-party membership and a Group Member Plan for up to six members. A company is allowed to purchase as many Group Member Plans as they need.

AutoBuy PRIMO Rewards Travel Members have access to the Rewards program professional concierge 24 hours a day and 365 days a year. AutoBuy PRIMO Rewards Travel Members can enjoy concierge-arranged domestic and international flights with one of the Rewards programs Shared Private Jet partner firms, a yacht at their destination, a villa rental, seaside village accommodations, and other expanded travel opportunities.

AutoBuy PRIMO PLUS Rewards Travel Memberships contain additional benefits including services with a Rewards authorized OEM Automotive Dealership partner to assist in the purchase and bidding process at any auto auction worldwide. Auto and vehicle equipment auctions are held worldwide and this service will allow a PRIMO PLUS member to bid without traveling to each auction venue. This service can also include the OEM Automotive Dealership partner assisting in the vetting of the auto or equipment, securing average pricing for like vehicles, transportation of purchased vehicles through transport industry partners, using a Pooling of Points (POP) program, and any number of other services.

A Rewards member's first annual Opt.In membership fee will be paid upon joining with the credit card that will connect with the AutoBuy Rewards Membership program. After the first annual fee, the membership annual fee can be automatically debited to the members' Personal Rewards Points Redemption Account with the member receiving an email showing the payment debited from the member's account. Rewards concierge travel contact can be with a professional concierge fluent in multiple languages.

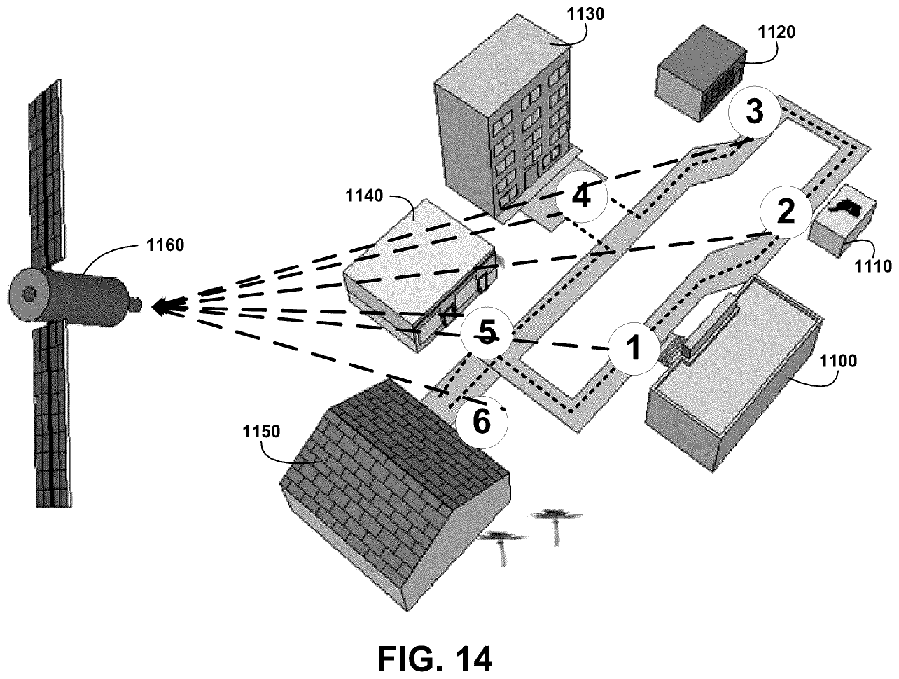

FIG. 14 shows for illustrative purposes only an example of tracking your visits of one embodiment. FIG. 14 shows the rewards program digital device application 400 is used for tracking opt-in subscribed member visits to destinations to rank their favorites and most frequent destinations. The rewards program digital device application 400 can use the GPS location of a member's smartphone 510 and satellite GPS locator 1160 systems to record daily activities including a trip to a #1 library 1100 to research a favorite subject, author, or genre of literature; #2 pharmacy 1110 to pick up a prescription that may be available through a contracted pharmacy company where the member can use rewards points to pay for the prescription; and #3 sporting goods store 1120 to pick up tennis equipment that may be available through a contracted sporting goods company where the member can redeem rewards points for the purchase. The opt-in subscribed member may visit a #4 doctor's office 1130 to consult on a health risk assessment analysis alert; then proceed to a #5 grocery store 1140 to pick up groceries for example that may include food and beverage items that may be available by the member redeeming rewards points; and finally returning to #6 home 1150 of one embodiment.

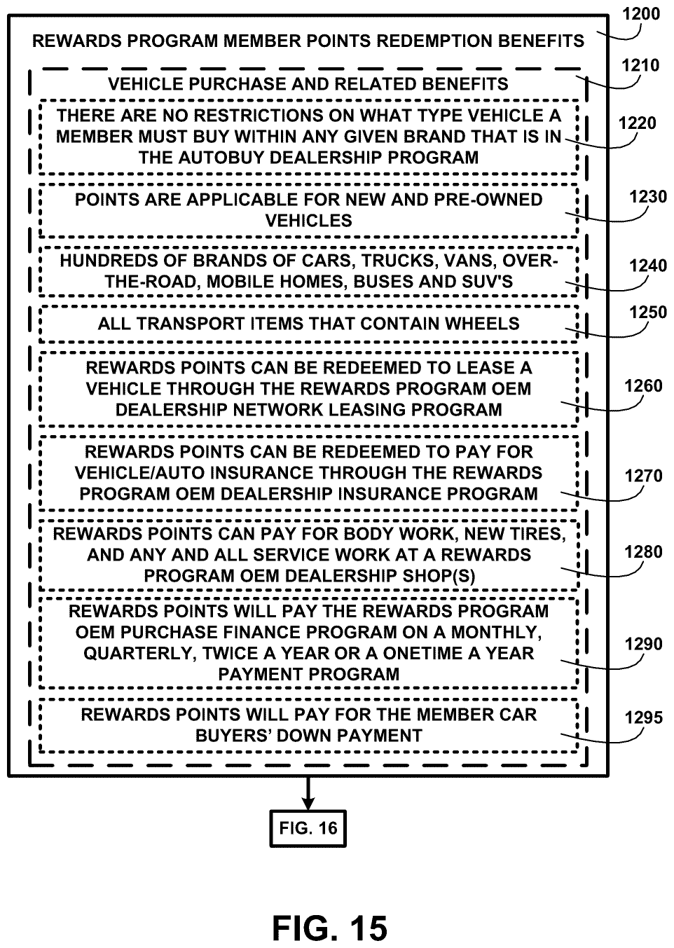

FIG. 15 shows a block diagram of an overview of AutoBuy earnest rewards program benefits of one embodiment. FIG. 15 shows AutoBuy earnest rewards program benefits which the member can subscribe to as one rewards program member points redemption benefits 1200 programs. The AutoBuy earnest rewards program benefits include a vehicle purchase and related benefits 1210. There are no restrictions on what type of vehicle a member must buy within any given brand that is in the AutoBuy dealership program 1220. Points are applicable for new and pre-owned vehicles 1230 including hundreds of brands of cars, trucks, vans, over-the-road, mobile homes, buses, and SUVs 1240 and all transport items that contain wheels 1250.

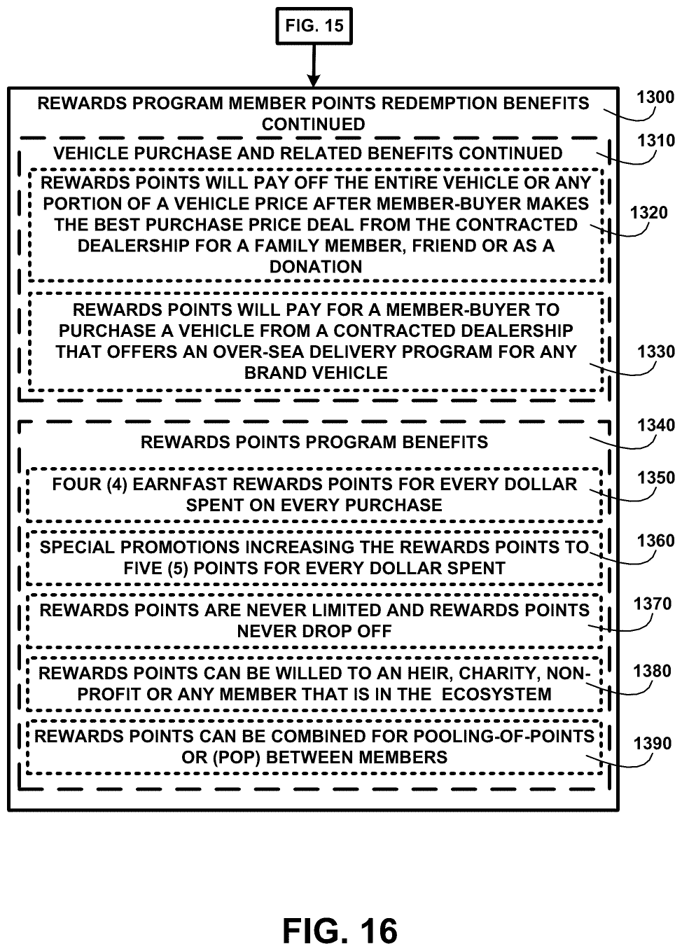

Rewards points can be redeemed to lease a vehicle through the rewards program OEM dealership network leasing program 1260. Rewards points can be redeemed to pay for vehicle/auto insurance through the rewards program OEM dealership insurance program 1270. Rewards points can pay for bodywork, new tires, and any service work at a rewards program OEM dealership shop(s) 1280. Rewards points will pay the rewards program OEM purchase finance program on a monthly, quarterly, twice a year, or a one-time a year payment program 1290. Rewards points will pay for the member car buyers' down payment 1295 of one embodiment. The descriptions continue in FIG. 16.

FIG. 16 shows a block diagram of an overview of vehicle purchase and related benefits of one embodiment. FIG. 16 shows continuing from FIG. 15 the rewards program member Rewards Points redemption benefits continued 1300 and vehicle purchase and related benefits continued 1310. Rewards points will pay off the entire vehicle or any portion of a vehicle price after the member-buyer makes the best purchase price deal from the contracted dealership for a family member, or friend or as a donation 1320. Rewards points will pay for a member-buyer to purchase a vehicle from a contracted dealership that offers an over-sea delivery program for any brand vehicle 1330. Rewards points program benefits 1340 include four (4) Earnfast rewards points for every dollar spent on every purchase 1350. Special promotions increase the rewards points to five (5) points for every dollar spent 1360. Rewards points are never limited and rewards points never drop off 1370. Rewards points can be willed to an heir, charity, non-profit, or any member that is in the Ecosystem 1380. Rewards points can be combined for Pooling-Of-Points or (POP) between members 1390 of one embodiment.

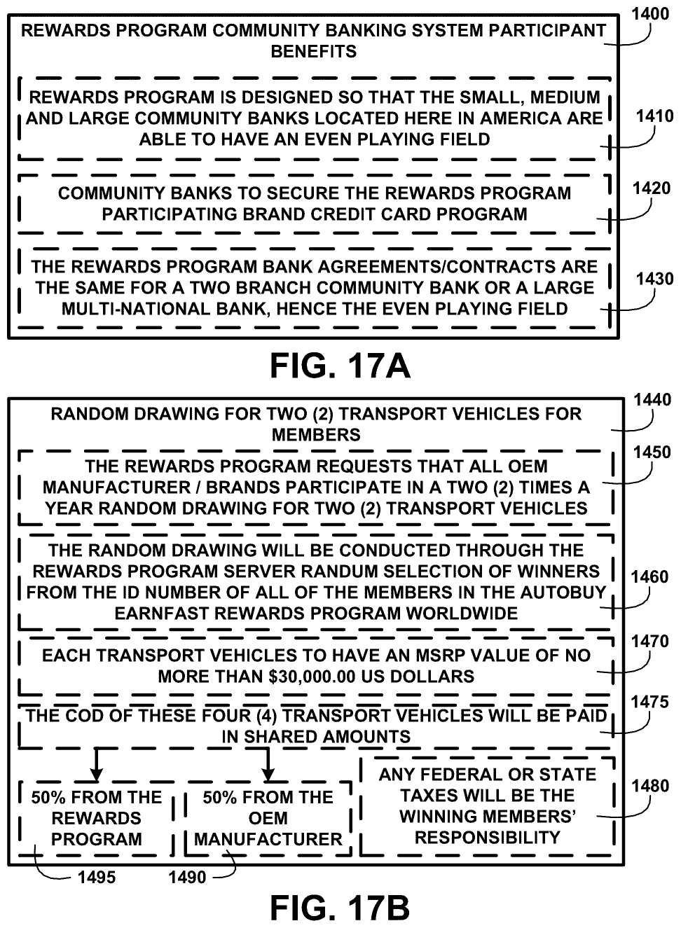

FIG. 17A shows a block diagram of an overview of community banking system participant benefits of one embodiment. FIG. 17A shows a rewards program community banking system participant benefits 1400. The rewards program is designed so that the small, medium, and large community banks located here in America can have an Even Playing Held 1410 for community banks to secure the rewards program participating brand credit card program 1420. The rewards program bank agreements/contracts are the same for a two-branch community bank or a large multi-national bank, hence the Even Playing Held 1430 of one embodiment.

FIG. 17B shows a block diagram of an overview of the random drawing for members of one embodiment. FIG. 17B shows a random drawing for two (2) transport vehicles for members 1440. The rewards program requests that all OEM manufacturers/brands participate in a two (2) times a year random drawing for two (2) transport vehicles 1450. The random drawing will be conducted through the rewards program server random selection of winners from the ID number of all of the members in the AutoBuy Earnfast Rewards Program worldwide 1460. Each transport vehicle has an MSRP value of no more than $30,000.00 US dollars 1470. The COD of these four (4) transport vehicles will be paid in shared amounts 1475 of 50% from the rewards program 1495 and 50% from the OEM manufacturer 1490. Any federal or state taxes will be the winning members' responsibility 1480 of one embodiment.

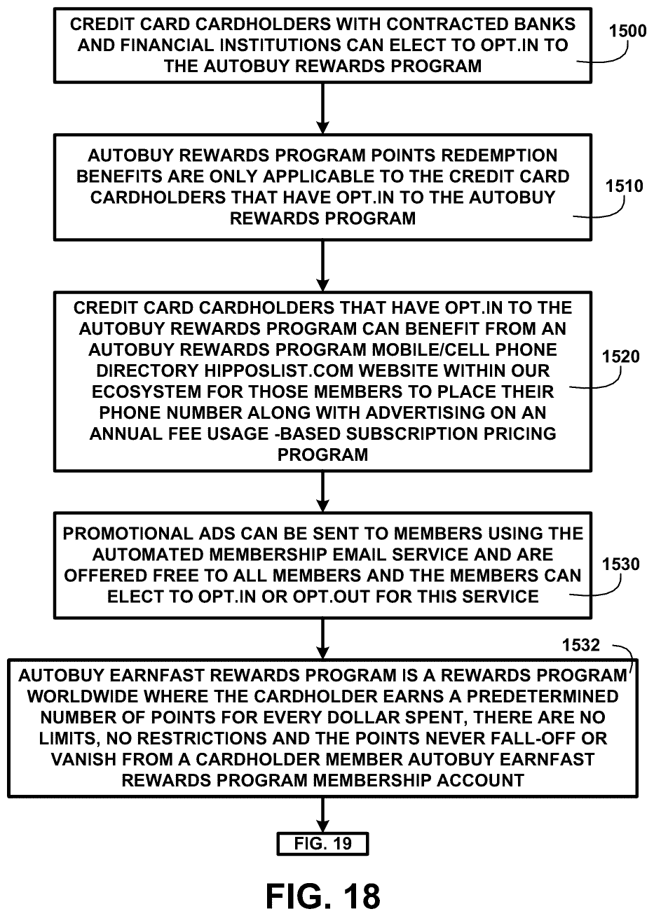

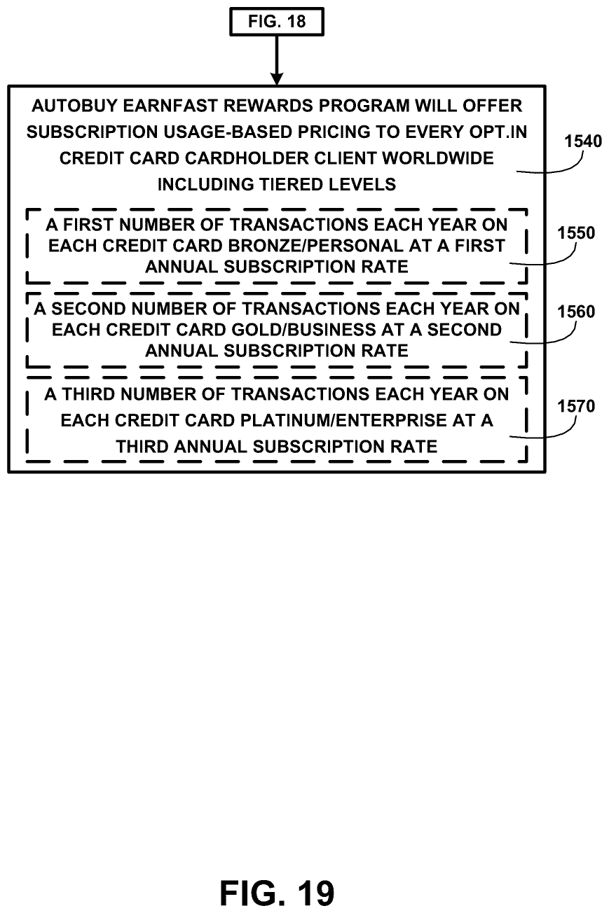

FIG. 18 shows a block diagram of an overview of Opt.In Ecosystem program of one embodiment. FIG. 18 shows credit card cardholders with contracted banks and financial institutions can elect to Opt.In to the AutoBuy Rewards program 1500. AutoBuy rewards program points redemption benefits are only applicable to credit card cardholders that have Opt.In to the AutoBuy Rewards program 1510. Credit card cardholders that have Opt.In to the AutoBuy Rewards program can benefit from an AutoBuy Rewards program mobile/cell phone directory Hipposlist.com website within our Ecosystem for those members to place their phone number along with advertising on an annual fee usage-based subscription pricing program 1520. Promotional ads can be sent to members using the automated membership email service and are offered free to all members and the members can elect to Opt.In or Opt.Out for this service 1530. AutoBuy Earnfast rewards program is a rewards program worldwide where the cardholder earns a predetermined number of points for every dollar spent, there are no limits, no restrictions, and the points never fall off or vanish from a cardholder member AutoBuy Earnfast rewards program membership account 1532 of one embodiment. Additional descriptions continue on FIG. 19.

FIG. 19 shows a block diagram of an overview of the AutoBuy Earnfast rewards program worldwide of one embodiment. FIG. 19 shows continuing from FIG. 18 that the AutoBuy Earnfast rewards program will offer subscription usage-based pricing to every Opt.In credit card cardholder client worldwide including tiered levels 1540. AutoBuy Earnfast rewards program subscription tiered levels include the first number of transactions each year on each credit card bronze/personal at a first annual subscription rate 1550; a second number of transactions each year on each credit card gold/business at a second annual subscription rate 1560; and a third number of transactions each year on each credit card platinum/enterprise at a third annual subscription rate 1570 of one embodiment.

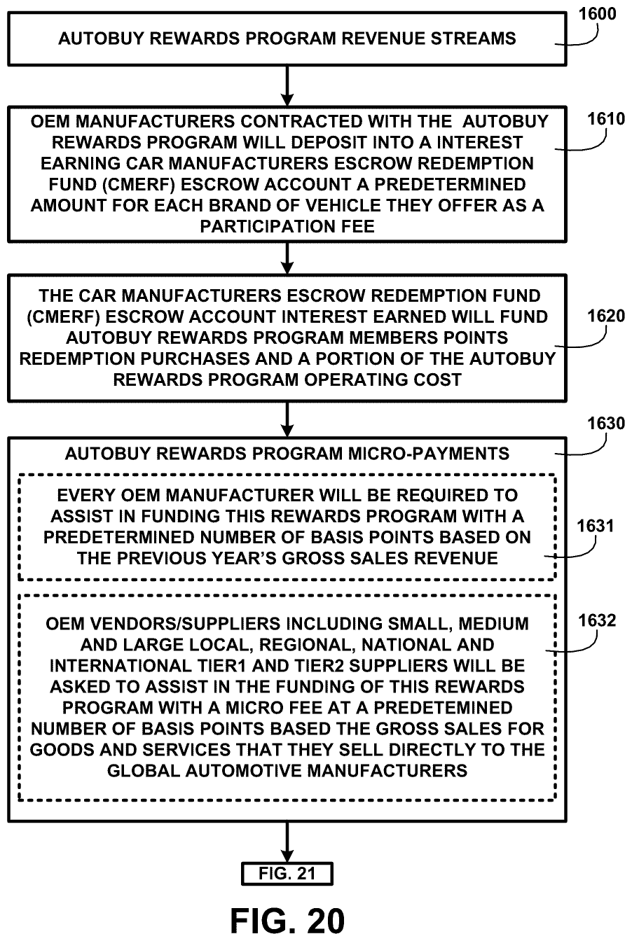

FIG. 20 shows a block diagram of an overview of the AutoBuy Rewards program revenue streams of one embodiment. FIG. 20 shows some embodiments of AutoBuy Rewards program revenue streams 1600. AutoBuy rewards program revenue streams 1600 can include a revenue stream where OEM manufacturers contracted with the AutoBuy Rewards program will deposit into an interest-earning Car Manufacturers Escrow Redemption Fund (CMERF) escrow account a predetermined amount for each brand of vehicle they offer as a participation fee 1610.

At least one rewards program server 500 of FIG. 8; at least one digital database 550 of FIG. 8; at least one digital processor 560 of FIG. 8 can be configured for recording and monitoring funding from contracted companies including interest earning from the Car Manufacturers Escrow Redemption Fund (CMERF) escrow account. The Car Manufacturers Escrow Redemption Fund (CMERF) escrow account interest earned will fund AutoBuy Rewards program members' points redemption purchases and a portion of the AutoBuy Rewards program operating cost 1620 of one embodiment.

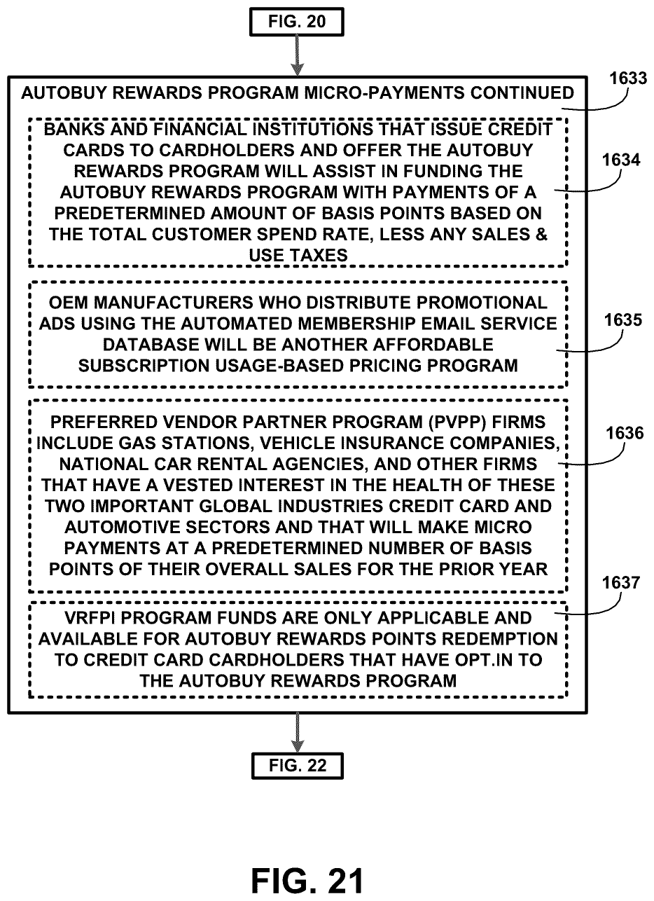

AutoBuy rewards program micro-payments 1630 are another embodiment of the revenue streams. Every OEM manufacturer will be required to assist in funding this rewards program with a predetermined number of basis points based on the previous year's gross sales revenue 1631. OEM vendors/suppliers including small, medium, and large local, regional, national, and international tier1 and tier2 suppliers will be asked to assist in the funding of this rewards program with a micro fee at a predetermined number of basis points based the gross sales for goods and services that they sell directly to the global automotive manufacturers 1632. Additional descriptions of micro-payments are in FIG. 21.

FIG. 21 shows a block diagram of an overview of the AutoBuy Rewards program micro-payments continued of one embodiment. FIG. 21 shows a continuation from FIG. 20 with AutoBuy Rewards program micro-payments continued 1633. Banks and financial institutions that issue credit cards to cardholders and offer the AutoBuy Rewards program will assist in funding the AutoBuy Rewards program with payments of a predetermined amount of basis points based on the total customer spend rate, less any sales & use taxes 1634. OEM manufacturers who distribute promotional ads using the automated membership email service database will be another affordable subscription usage-based pricing program 1635.

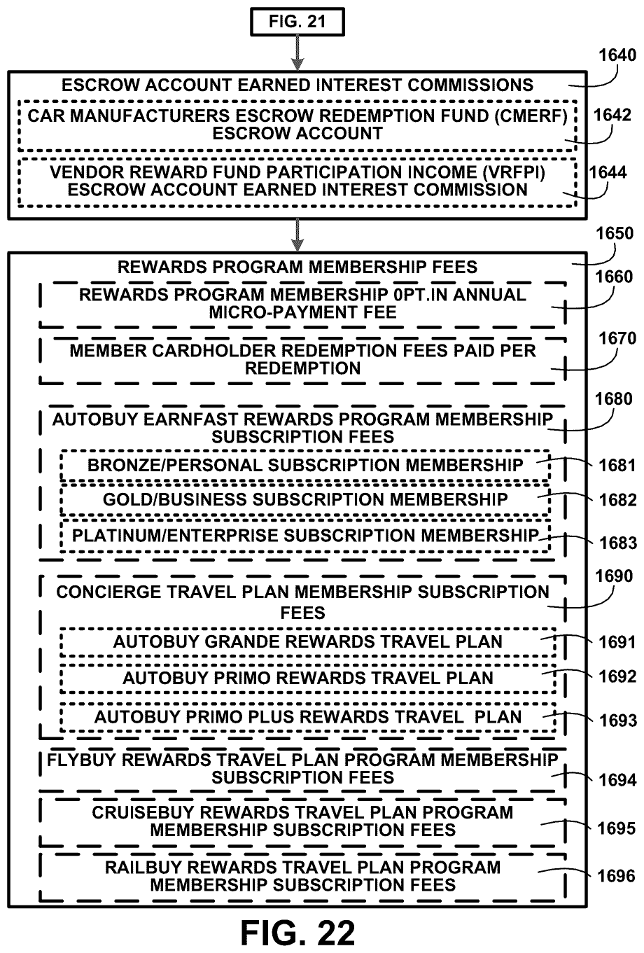

Preferred Vendor Partner Program (PVPP) firms include gas stations, vehicle insurance companies, national car rental agencies, and other firms that have a vested interest in the health of these two important global industries credit card and automotive sectors, and that will make micropayments at a predetermined number of basis points of their overall sales for the prior year 1636. VRFPI program funds are only applicable and available for AutoBuy Rewards points redemption to credit card cardholders that have Opt.In to the AutoBuy Rewards program 1637 of one embodiment. Descriptions of more revenue streams continue in FIG. 22.

FIG. 22 shows a block diagram of an overview of escrow account earned interest commissions of one embodiment. FIG. 22 shows a continuation from FIG. 21 of revenue streams with escrow account earned interest commissions 1640. Escrow account earned interest commissions 1640 include a predetermined percentage of the interest earned from the Car Manufacturers Escrow Redemption Fund (CMERF) escrow account 1642 and the Vendor Reward Fund Participation Income (VRFPI) escrow account earned interest commission 1644. At least one rewards program server 500 of FIG. 8; at least one digital database 550 of FIG. 8; at least one digital processor 560 of FIG. 8 can be configured for recording and monitoring funding from contracted companies including interest earning from the Vendor Reward Fund Participation Income (VRFPI) escrow account of one embodiment.

Rewards program membership fees 1650 are another source of revenue streams. Rewards program membership fees 1650 include a Rewards program membership of 0pt.in annual micro-payment fee 1660. Another revenue source is Member cardholder redemption fees paid per redemption 1670. AutoBuy Earnfast rewards program membership subscription fees 1680 including the Bronze/personal subscription membership 1681; Gold/business subscription membership 1682; and Platinum/enterprise subscription membership 1683. Another revenue stream is generated with Concierge travel plan membership subscription fees 1690 including an AutoBuy GRANDE rewards travel plan 1691; AutoBuy PRIMO rewards travel plan 1692; and AutoBuy PRIMO PLUS rewards travel plan 1693. Yet another revenue stream is generated with flybuy rewards travel plan program membership subscription fees 1694, cruisebuy rewards travel plan program membership subscription fees 1695, and railbuy rewards travel plan program membership subscription fees 1696 of one embodiment.

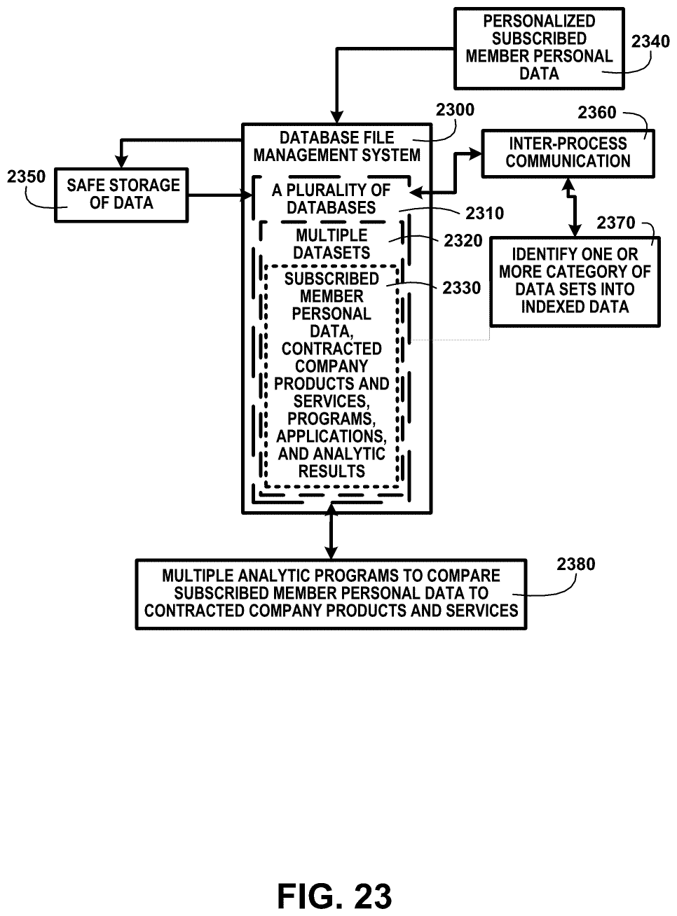

FIG. 23 shows a block diagram of an overview of a plurality of databases of one embodiment. FIG. 23 shows a database file management system 2300 to regulate the processes of a plurality of databases 2310. A database manager of the database file management system is configured to implement security programs to ensure the safety of database stored data on the plurality of databases. The plurality of databases stores multiple datasets 2320. The multiple datasets include subscribed member personal data, contracted company products and services, programs, applications, and analytic results 2330.

The database file management system comprises a plurality of databases configured to store data. A network computer coupled to the plurality of databases is configured to gather personalized user data including user personal information, user transactional purchases, and user self-reported information for storage on the plurality of databases. A database schema coupled to the plurality of databases is configured to categorize the personalized user data. An inter-process communication processor coupled to the plurality of databases is configured to determine potential third-party redemption benefits for the user. A program redemption points program, wherein a user can earn program redemption points with transactional purchases using a contracted credit card. A user can make transactional purchases of the customized third-party redemption benefits with the earned redemption points.

The inter-process communication processor is further configured to assign the customized third-party redemption benefits to the user based on the categorized personalized user data of the database schema.

Inter-process communication 2360 devices of the database manager coupled to the plurality of databases processes personalized subscribed member personal data 2340. Multiple analytic programs to compare subscribed member personal data to contracted company products and services 2380 to display products and services to the subscribed member that are matched to the subscribed personalized likes. The Inter-process communication 2360 devices identify one or more category of data sets into indexed data 2370 within the safe storage of data 2350 of one embodiment.

The foregoing has described the principles, embodiments, and modes of operation of the embodiments. However, the embodiments should not be construed as being limited to the particular embodiments discussed. The above-described embodiments should be regarded as illustrative rather than restrictive, and it should be appreciated that variations may be made in those embodiments by workers skilled in the art without departing from the scope of the present invention as defined by the following claims.

What is claimed is:

1. A database file management system for an interactive artificial intelligence personalized rewards system, comprising:
a plurality of databases configured to store data;
an interactive artificial intelligence and machine learning processor configured to receive and analyze personalized user data to determine likes and dislikes of a user to constantly create and update information about the user;
a network computer coupled to the plurality of databases configured to gather the personalized user data including user personal information, user transactional purchases, individual preferences, browsing patterns, social media interactions of the user and self-reported information for storage on the plurality of databases;
a database schema coupled to the plurality of databases configured to categorize the personalized user data;
a mobile device with a camera configured to capture images of at least one document with health related information about the user and to convert the captured images into machine readable text using an optical character recognition processor;
an inter-process communication processor coupled to the plurality of databases configured to analyze the machine readable text to create a health risk assessment of the user and to analyze the health risk assessment and the personalized user data to determine potential third-party redemption benefits for the user; and
wherein the inter-process communication processor is further configured to assign customized third-party redemption benefits to the user based on the categorized personalized user data of the database schema.

2. The database file management system for an interactive artificial intelligence personalized rewards system of claim 1, wherein the at least one document with the health related information includes ancestry DNA tests.

3. The database file management system for an interactive artificial intelligence personalized rewards system of claim 1, wherein the plurality of databases includes data storage of personalized user data, contracted third-party products and services, programs, applications, and analytic results.

4. The database file management system for an interactive artificial intelligence personalized rewards system of claim 1, further comprising a graphical user interface configured to allow a user to make transactional purchases of the customized third-party redemption benefits with earned redemption points.

5. The database file management system for an interactive artificial intelligence personalized rewards system of claim 1, wherein the database schema is further configured to determine potential third-party redemption benefits that are selectable by the user to make a transactional purchase of the customized third-party redemption benefits with earned redemption points.

6. The database file management system for an interactive artificial intelligence personalized rewards system of claim 1, wherein the plurality of databases is further configured to store the customized third-party redemption benefits on the plurality of databases.

7. The database file management system for an interactive artificial intelligence personalized rewards system of claim 1, wherein the inter-process communication processor is further configured to determine potential third-party redemption benefits for the user from contracted third-party products and services.

8. The database file management system for an interactive artificial intelligence personalized rewards system of claim 1, wherein the inter-process communication processor is further configured to allow the user to earn program redemption points with user credit card transactional purchases.

9. The database file management system for an interactive artificial intelligence personalized rewards system of claim 1, further comprising a graphical user interface configured to allow the user to select from the potential third-party redemption benefits.

10. The database file management system for an interactive artificial intelligence personalized rewards system of claim 1, wherein the inter-process communication processor is further configured to index personalized user data for future queries.

11. A database file management system for an interactive artificial intelligence personalized rewards system, comprising:
a plurality of databases configured to store data;
an interactive artificial intelligence and machine learning processor configured to receive and analyze personalized user data to determine likes and dislikes of a user to constantly create and update information about the user;
a network computer coupled to the plurality of databases configured to gather the personalized user data including user personal information, user transactional purchases, individual preferences, browsing patterns, social media interactions of the user and self-reported information for storage on the plurality of databases;
a database schema coupled to the plurality of databases configured to categorize the personalize user data;
a mobile application operating on a smart phone having a camera configured to capture images of at least one document with ancestry DNA related information about the user and to convert the captured images into machine readable text using an optical character recognition processor;
an inter-process communication processor coupled to the plurality of databases configured to analyze the machine readable text to create a health risk assessment of the user and to analyze the health risk assessment and the personalized user data to determine potential third-party redemption benefits for the user;
wherein the inter-process communication processor is further configured to assign customized third-party redemption benefits to the user based on the categorized personalized user data of the database schema; and
a program schema configured to store in the plurality of databases earned benefit points of the user that are associated with the customized third-party redemption benefits, wherein the user is allowed redeem the benefit points stored in the plurality of databases for goods and services.

12. The database file management system for an interactive artificial intelligence personalized rewards system of claim 11, wherein the subscribed member's health risk assessment includes a heart condition analysis.

13. The database file management system for an interactive artificial intelligence personalized rewards system of claim 11, wherein the inter-process communication processor is further configured to index the personalized user data to categorize the personalized user data and match program points redemption benefits to the personalized user data categories.

14. The database file management system for an interactive artificial intelligence personalized rewards system of claim 11, wherein the inter-process communication processor is further configured to allow a user to earn the program redemption points using a contracted user credit card for the user transactional purchases.

15. A database file management system for an interactive artificial intelligence personalized rewards system, comprising:
an interactive artificial intelligence and machine learning processor configured to receive and analyze personalized user data to determine likes and dislikes of a user to constantly create and update information about the user;
a plurality of databases configured to gather the personalized user data including user personal information, user transactional purchases, individual preferences, browsing patterns, social media interactions of the user and self-reported information for storage on the plurality of databases;
a database schema coupled to the plurality of databases configured to categorize the personalize user data;
a mobile device with a camera configured to capture images of at least one document with health related information about the user and to convert the captured images into machine readable text using an optical character recognition processor;
an inter-process communication processor coupled to the plurality of databases configured to analyze the machine readable text to create a health risk assessment of the user and to analyze the health risk assessment and the personalized user data to determine potential third-party redemption benefits for the user;
wherein the inter-process communication processor is further configured to assign customized third-party redemption benefits to the user based on the categorized personalized user data of the database schema;
a program schema configured to store in the plurality of databases earned benefit points of the user that are associated with the customized third-party redemption benefits, wherein the user is allowed redeem the benefit points stored in the plurality of databases for goods and services; and
a graphical user interface configured to allow the user to redeem the benefit points stored in the plurality of databases.

16. The database file management system for an interactive artificial intelligence personalized rewards system of claim 15, wherein the plurality of databases is further configured to store the categorized personalized user data on the plurality of databases.

17. The database file management system for an interactive artificial intelligence personalized rewards system of claim 15, wherein the inter-process communication processor is further configured to compare the personalized user data to contracted third-party products and services to determine potential customized redemption benefits.

18. The database file management system for an interactive artificial intelligence personalized rewards system of claim 15, further comprising a server configured to transmit the health risk assessment analysis results to the subscribed member with early health risks alerts.

19. The database file management system for an interactive artificial intelligence personalized rewards system of claim 15, further comprising a transaction manager coupled to the plurality of databases configured to process one or more database access operations.

20. The database file management system for an interactive artificial intelligence personalized rewards system of claim 15, further comprising a query optimizer coupled to the plurality of databases configured to select a plan with the lowest time to provide a response to a query of the personalized user data.
